(19) United States
(12) Patent Application Publication
(10) Pub. No.: US 2025/0312299 A1
(43) Pub. Date: Oct. 9, 2025

(54) COMPOSITION COMPRISING THIOUREA DERIVATIVE FOR PREVENTION OR TREATMENT OF FIBROTIC DISEASE

(71) Applicant: THERASID BIOSCIENCE INC., Seongnam-si, Gyeonggi-do (KR)

(72) Inventors: Heung Jae KIM, Seongnam-si, Gyeonggi-do (KR); Sung Ki SEO, Seoul (KR); Woo Young KWAK, Yongin-si, Gyeonggi-do (KR); Jae Min OH, Seoul (KR); Dong Uk LIM, Seoul (KR); Jae Hyuk LEE, Incheon (KR); Bo Yeon KANG, Gunpo-si, Gyeonggi-do (KR)

(21) Appl. No.: 18/863,997

(22) PCT Filed: May 8, 2023
(86) PCT No.: PCT/KR2023/006217

§ 371 Date: Nov. 7, 2024

(30) Foreign Application Priority Data
May 9, 2022 (KR) 10-2022-0056757

(51) Int. Cl. A61K 31/17; A61P 1/16; A61P 11/00; A61P 13/12

(52) U.S. Cl. CPC A61K 31/17; A61P 1/16; A61P 11/00; A61P 13/12

(57) ABSTRACT

The present invention relates to a composition comprising the compound of chemical formula 1 or a pharmaceutically acceptable salt thereof as an active ingredient for the prevention or treatment of fibrotic diseases. The composition according to the present invention inhibits the expression of collagen type 1 (COL1A1) or fibronectin and thus can be advantageously used as a composition for the prevention or treatment of fibrotic diseases.

### TECHNICAL FIELD

The present invention relates to a novel use of a thiourea derivative. Specifically, the present invention relates to a composition for the prevention or treatment of fibrosis, comprising the compound of chemical formula 1 below or a pharmaceutically acceptable salt thereof as an active ingredient.

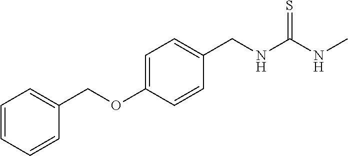 [SMILES: CNC(=S)NCc1ccc(OCc2ccccc2)cc1]

### BACKGROUND ART

Fibrosis refers to a phenomenon in which fibrous connective tissue is excessively formed in an organ. Fibrosis is a phenomenon similar to the formation of scar tissue seen in the process of recovering from a wound, but can paralyze the normal structure and function of the organ. That is, extracellular matrix such as collagen is secreted by activated fibroblasts. One of the well-known initial processes begins with the secretion of signaling substances that activate fibroblasts by immune cells including macrophages. A representative signaling substance is transforming growth factor-beta, and fibroblasts that receive the signal are activated along with proliferation and begin to secrete extracellular matrix.

Fibrosis can be seen as the final result of a response to persistent infection, autoimmune response, allergic response, exposure to chemical substances and radiation, and physical injury. Although it is an essential phenomenon for recovery from these stimuli, but if the stimuli occur repeatedly or severely or the fibrosis process is not controlled, it can lead to pathological phenomena and may be irreversible. Pulmonary fibrosis and liver fibrosis are representative fibrotic diseases.

Pulmonary fibrosis is a phenomenon in which fibrosis occurs in the lungs due to environmental factors, infection, or unknown causes. In the case of idiopathic pulmonary fibrosis of unknown cause, some genetic basis has been suggested. Mutations in the surfactant protein gene have been found in some patients with a family history, and mutations in telomerase have also been reported. Symptoms of pulmonary fibrosis include shortness of breath, dry cough, and clubbing of the nails, and there is still no treatment method for the lung tissue that has eventually lost elasticity.

A representative case of liver fibrosis is cirrhosis. Although it can be idiopathic, it is known to be mainly induced by alcoholism, hepatitis B, hepatitis C, or fatty liver. Ascites is a common symptom, and jaundice and bleeding may also occur. Cirrhosis can be prevented through vaccination in cases such as hepatitis B, and can be improved to some extent through abstinence from alcohol and weight loss, but it is an irreversible phenomenon when it progresses to the terminal stage, and liver transplantation is the only treatment method.

In addition, kidney fibrosis is a representative tissue finding in chronic kidney disease, and appears as a result of abnormal deposition of the extracellular matrix. This is the result of an increase, deposition, and structural stabilization of the extracellular matrix due to changes in the enzymes that regulate the extracellular matrix. Treatment of kidney fibrosis uses drugs that have the effect of suppressing the production of angiotensin, but has side effects such as dizziness, back pain, fatigue, and coughing. Furthermore, once kidney fibrosis has fully progressed, there is no appropriate treatment method other than kidney transplantation.

Under this background, the present inventors have confirmed through research on thiourea derivatives known as activators of RORα that thiourea derivatives suppress the expression of fibrosis markers in various cells, thereby discovering that they can be applied as inhibitors of fibrosis. Based on the above, the present inventors have completed the present invention.

### PRIOR ART DOCUMENT

### Patent Document

(Patent Document 1) Korean Patent Registration No. 10-1450960
(Patent Document 2) U.S. Patent Application Publication No. 2021-0155586

### DETAILED DESCRIPTION OF INVENTION

### Technical Problem

An object of the present invention is to provide a pharmaceutical composition for the prevention or treatment of fibrotic diseases, comprising the compound of chemical formula 1 below or a pharmaceutically acceptable salt thereof as an active ingredient.

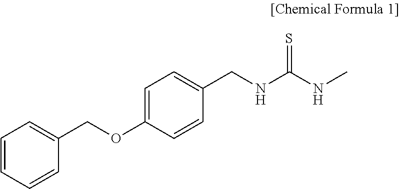 [SMILES: CNC(=S)NCc1ccc(OCc2ccccc2)cc1]

In addition, another object of the present invention is to provide a method for treating a patient having a fibrotic disease, comprising the compound of chemical formula 1 or a pharmaceutically acceptable salt thereof as an active ingredient.

### Solution to Problem

The present invention provides a pharmaceutical composition for the prevention or treatment of fibrosis, comprising the compound of chemical formula 1 below or a pharmaceutically acceptable salt thereof as an active ingredient.

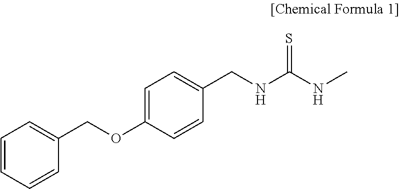 [SMILES: CNC(=S)NCc1ccc(OCc2ccccc2)cc1]

In addition, the present invention provides a method for treating a patient having a fibrotic disease, comprising the compound of chemical formula 1 or a pharmaceutically acceptable salt thereof as an active ingredient.

The fibrosis may be selected from the group consisting of fibrosis of the lung, kidney, liver, heart, brain, blood vessels, joints, intestines, skin, soft tissue, bone marrow, penis, peritoneum, muscle, spine, testis, ovary, breast, thyroid, eardrum, pancreas, gallbladder, bladder, and prostate, and may preferably be liver fibrosis, pulmonary fibrosis, or kidney fibrosis.

The composition can prevent or treat fibrosis by inhibiting the expression of collagen type 1 (COL1A1) or inhibiting the expression of fibronectin.

The present invention provides a composition for the prevention or treatment of fibrosis, comprising the compound of chemical formula 1 or a pharmaceutically acceptable salt thereof as an active ingredient.

The present invention provides a use of the composition comprising the compound of chemical formula 1 or a pharmaceutically acceptable salt thereof as an active ingredient for the prevention or treatment of fibrosis.

The present invention provides a method for preventing or treating fibrosis by administering the composition comprising the compound of chemical formula 1 or a pharmaceutically acceptable salt thereof as an active ingredient to a subject having a fibrotic disease.

### Effects of Invention

The compound of chemical formula 1 according to the present invention not only does not exhibit cytotoxicity in liver, lung, and kidney cells, but also inhibits the expression of collagen type 1 (COL1A1) or fibronectin, which are fibrosis-related markers, and thus can be advantageously used as a composition for the prevention or treatment of fibrosis.

### BRIEF DESCRIPTION OF DRAWINGS

FIG. 1 shows the cell viability according to compound treatment in liver cells (LX2).

FIG. 2 shows the cell viability according to compound treatment in lung cells (A549).

FIG. 3 shows the cell viability according to compound treatment in kidney cells (HK-2).

FIGS. 4 and 5 show the gene expression levels of COL1A1 and fibronectin according to compound treatment in liver cells (LX2).

FIGS. 6 and 7 show the gene expression levels of COL1A1 and fibronectin according to compound treatment in lung cells (A549).

FIGS. 8 and 9 show the gene expression levels of COL1A1 and fibronectin according to compound treatment in kidney cells (HK-2).

FIGS. 10 to 12 show the protein expression levels of COL1A1, fibronectin, and HSP60 according to compound treatment in liver cells (LX2).

FIGS. 13 to 15 show the protein expression levels of COL1A1, fibronectin, and HSP60 according to compound treatment in lung cells (A549).

FIGS. 16 to 18 show the protein expression levels of COL1A1, fibronectin, and HSP60 according to compound treatment in kidney cells (HK-2).

### BEST MODE FOR CARRYING OUT THE INVENTION

Hereinafter, with reference to the accompanying drawings, aspects and examples of the present disclosure will be described in detail so that those of ordinary skill in the art to which the present invention belongs can easily carry out the present invention. However, the present disclosure may be implemented in various forms and is not limited to the aspects and examples described herein.

Throughout the present specification, when a certain part “comprises” a certain element, it means that other elements may be further comprised, rather than excluding other elements, unless otherwise stated.

The present invention provides a composition for preventing, ameliorating, or treating fibrosis, comprising the compound of chemical formula 1 below, and the composition may be a pharmaceutical composition, a food composition, or an animal feed composition.

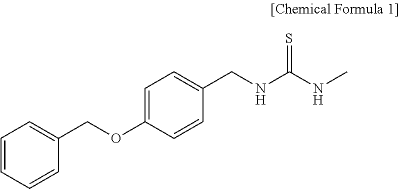 [SMILES: CNC(=S)NCc1ccc(OCc2ccccc2)cc1]

The compound of chemical formula 1 above is a thiourea derivative, ‘1-(4-benzyloxy-benzyl)-3-methyl-thiourea’ (hereinafter also referred to as ‘TB-840’), and the method for preparing the compound is described in Korean Patent Registration No. 10-1450960, which is incorporated herein by reference.

The composition can inhibit the expression of collagen type 1 (type 1 collagen; COL1A1) or the expression of fibronectin, and the expression of COL1A1 and fibronectin includes the expression of a gene or protein.

In the present invention, ‘fibrosis’ refers to a symptom in which a tissue loses its function due to fibrosis in which normal tissue is destroyed and replaced with fibrous connective tissue. According to an embodiment of the present invention, the fibrosis may be liver fibrosis, pulmonary fibrosis, or kidney fibrosis, but is not limited thereto.

The liver fibrosis refers to a symptom in which liver tissue hardens due to repeated damage and recovery of the liver, and pulmonary fibrosis refers to a disease in which lung tissue cells change into fibrocytes, causing symptoms such as shortness of breath, coughing, cyanosis, and clubbing, and kidney fibrosis refers to a symptom in which kidney tissue becomes fibrotic due to causes such as excessive inflammatory response occurring in kidney tissue and fibrocytosis of epithelial cells, resulting in loss of kidney function.

As used herein, the term “prevention” refers to any action that inhibits or delays a disease and the like by administration of the composition according to the present invention, and “treatment” refers to any action that improves or beneficially changes the symptoms of the subject who is suspected to have or has developed a disease by administration of the composition. As used herein, the term “amelioration” refers to any action that at least reduces a parameter related to a condition to be treated by administration of a composition comprising the extract of the present invention, for example, the severity of symptoms.

Hereinafter, the present invention will be described in more detail through examples. However, these examples are provided for illustrative purposes only and the scope of the present invention is not limited to these examples.

### Example 1

### Evaluation of Cytotoxicity in Liver, Lung, and Kidney Cell Lines

In order to confirm whether the compound of the present invention exhibits cytotoxicity, the cell viability according to the treatment of the compound of the present invention in a liver cell line (LX2), a lung cell line (A549), and a kidney cell line (HK-2) was confirmed through a CCK-8 assay.

### 1-1. Liver Cell Line (LX2)

LX2 cells, which are liver cells (stellate cells), were seeded at 1×10⁴ per well in a 96 well plate, cultured for 24 hours in a 37° C., 5% CO₂ incubator, and then treated with the compound of the present invention (TB-840) at a concentration of 20 μM for 24 hours. After culturing for 24 hours, the cells were treated with 10 μl of CCK-8 solution for 2 hours. Thereafter, the 450 nm wavelength value was measured using a microplate reader to evaluate whether it exhibits cytotoxicity.

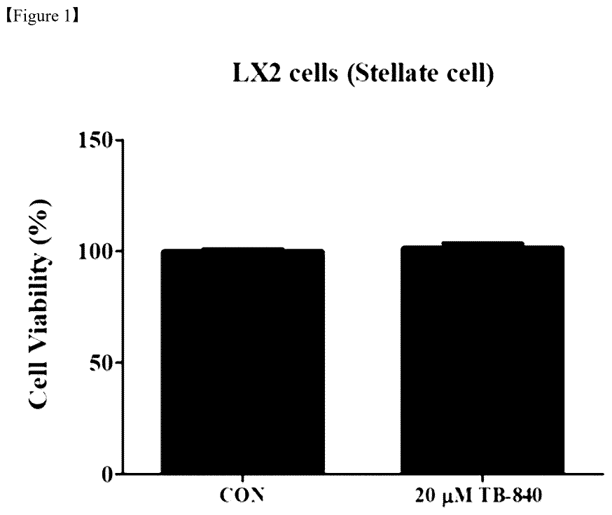

As a result, as shown in FIG. 1, the compound of the present invention was found to maintain cell viability at almost 100%, thereby confirming that the compound of the present invention is safe as it has no cytotoxicity.

### 1-2. Lung Cell Line (A549)

A549 cells, which are lung cells (lung carcinoma cells), were seeded at 1.5×10⁴ per well in a 96 well plate, cultured for 24 hours in a 37° C., 5% CO₂ incubator, and then treated with the compound of the present invention (TB-840) at a concentration of 20 μM for 24 hours. After culturing for 24 hours, the cells were treated with 10 μl of CCK-8 solution for 2 hours. Thereafter, the 450 nm wavelength value was measured using a microplate reader to evaluate whether it exhibits cytotoxicity.

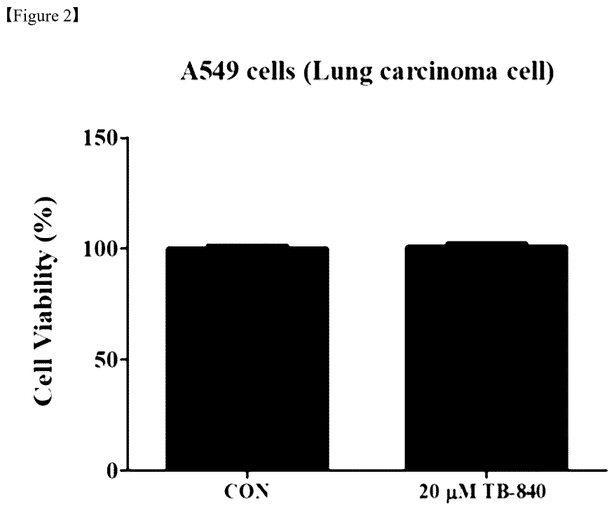

As a result, as shown in FIG. 2, the compound of the present invention was found to maintain cell viability at almost 100%, thereby confirming that the compound of the present invention is safe as it has no cytotoxicity.

### 1-3. Kidney Cell Line (HK-2)

HK-2 cells, which are kidney cells (proximal tubular cells), were seeded at 1×10⁴ per well in a 96 well plate, cultured for 24 hours in a 37° C., 5% CO₂ incubator, and then treated with the compound of the present invention (TB-840) at a concentration of 20 μM for 24 hours. After culturing for 24 hours, the cells were treated with 10 μl of CCK-8 solution for 2 hours. Thereafter, the 450 nm wavelength value was measured using a microplate reader to evaluate whether it exhibits cytotoxicity.

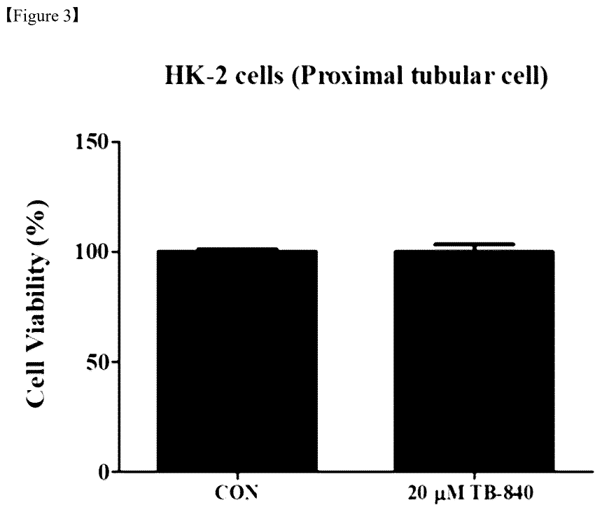

As a result, as shown in FIG. 3, the compound of the present invention was found to maintain cell viability at almost 100%, thereby confirming that the compound of the present invention is safe as it has no cytotoxicity.

### Example 2

### Confirmation of Inhibitory Effect on Gene Expression of Fibrosis-Related Markers in Liver, Lung, and Kidney Cell Lines

In order to confirm the inhibitory effect of the compound of the present invention on the gene expression of fibrosis-related markers, the gene expression of collagen type 1 (type 1 collagen; COL1A1) and fibronectin, which are fibrosis-related markers, was confirmed through RT-qPCR analysis. As a control group, TGF-β (transforming growth factor-β) was used. TGF-β is known to promote the production of extracellular matrix such as collagen and promote fibrosis of tissues.

### 2-1. Liver Cell Line (LX2)

LX2 cells were seeded at 3.5×10⁵ per well in a 6 well plate, cultured for 24 hours in a 37° C., 5% CO₂ incubator, and then treated with TGF-β at a concentration of 10 ng/ml and the compound of the present invention (TB-840) at a concentration of 20 μM, and cultured for 24 hours. After culturing for 24 hours, the cells were washed twice with phosphate buffered saline (PBS), and then RNA extraction was performed using a RNA-spin™ total RNA extraction kit (iNtRON Biotechnology, Cat no. 17211) protocol. Thereafter, the RNA concentration was measured using a Nanodrop device, and cDNA was synthesized using a Maxime™ RT PreMix (iNtRON Biotechnology, Cat no. 25081) and a MiniAmp™ Termal Cycler device. Thereafter, the gene expression levels of COL1A1 and fibronectin were measured using a QuantStudio 5 Real-Time PCR device.

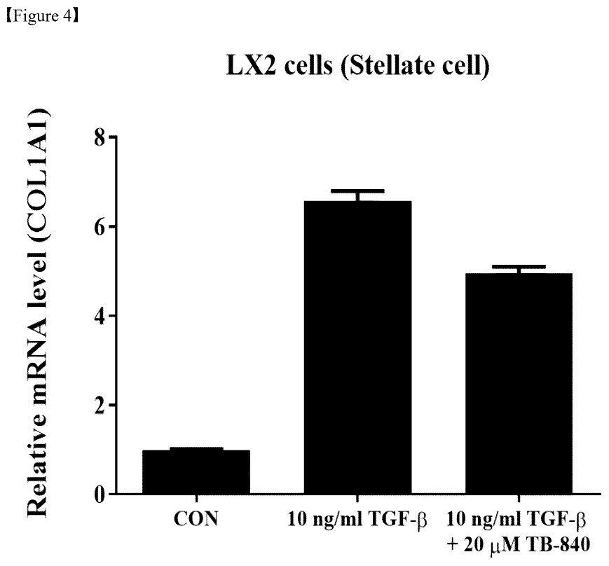

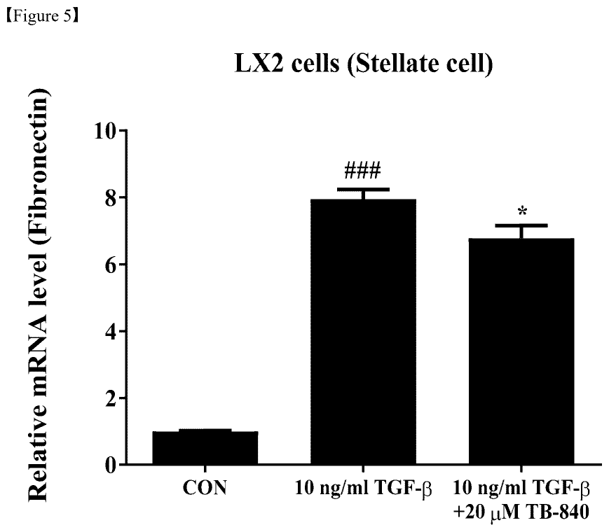

As a result, as shown in FIG. 4, the compound of the present invention was found to have reduced the gene expression of COL1A1 compared to the group treated with TGF-β at a concentration of 10 ng/ml, and as shown in FIG. 5, it was also found to have reduced the gene expression of fibronectin compared to the group treated with TGF-β at a concentration of 10 ng/ml.

Therefore, it was confirmed that the compound of the present invention inhibits the gene expression of fibrosis-related markers in liver cells, and through this, it can be seen that it has an inhibitory effect on liver fibrosis.

### 2-2. Lung Cell Line (A549)

A549 cells were seeded at 2.5×10⁵ per well in a 6 well plate, cultured for 24 hours in a 37° C., 5% CO₂ incubator, and then treated with TGF-β at a concentration of 10 ng/ml and the compound of the present invention (TB-840) at a concentration of 20 μM, and cultured for 24 hours. After culturing for 24 hours, the cells were washed twice with phosphate buffered saline (PBS), and then RNA extraction was performed using a RNA-spin™ total RNA extraction kit (iNtRON Biotechnology, Cat no. 17211) protocol. Thereafter, the RNA concentration was measured using a Nanodrop device, and cDNA was synthesized using a Maxime™ RT PreMix (iNtRON Biotechnology, Cat no. 25081) and a MiniAmp™ Termal Cycler device. Thereafter, the gene expression levels of COL1A1 and fibronectin were measured using a QuantStudio 5 Real-Time PCR device.

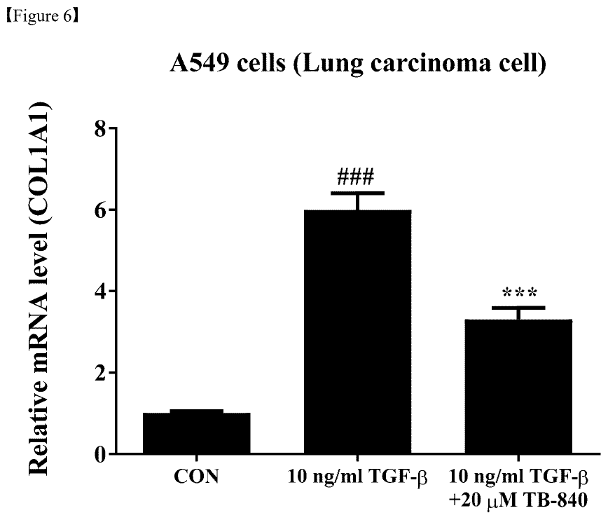

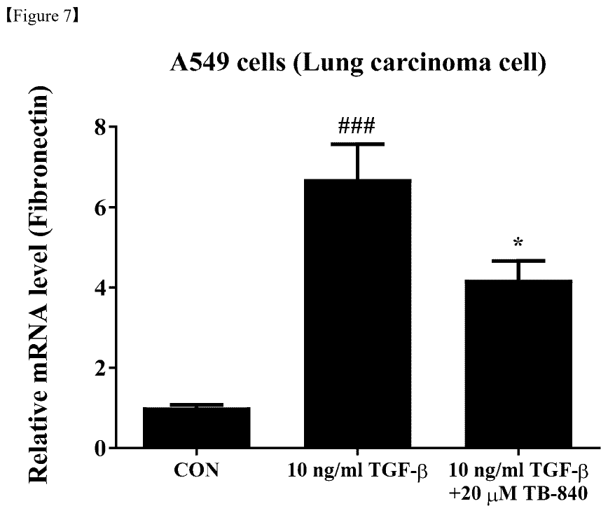

As a result, as shown in FIG. 6, the compound of the present invention was found to have reduced the gene expression of COL1A1 compared to the group treated with TGF-β at a concentration of 10 ng/ml, and as shown in FIG. 7, it was also found to have reduced the gene expression of fibronectin compared to the group treated with TGF-β at a concentration of 10 ng/ml.

Therefore, it was confirmed that the compound of the present invention inhibits the gene expression of fibrosis-related markers in lung cells, and through this, it can be seen that it has an inhibitory effect on pulmonary fibrosis.

### 2-3. Kidney Cell Line (HK-2)

HK-2 cells were seeded at 2×10⁵ per well in a 6 well plate, cultured for 24 hours in a 37° C., 5% CO₂ incubator, and then treated with TGF-β at a concentration of 10 ng/ml and the compound of the present invention (TB-840) at a concentration of 20 μM, and cultured for 24 hours. After culturing for 24 hours, the cells were washed twice with phosphate buffered saline (PBS), and then RNA extraction was performed using a RNA-spin™ total RNA extraction kit (iNtRON Biotechnology, Cat no. 17211) protocol. Thereafter, the RNA concentration was measured using a Nanodrop device, and cDNA was synthesized using a Maxime™ RT PreMix (iNtRON Biotechnology, Cat no. 25081) and a MiniAmp™ Termal Cycler device. Thereafter, the gene expression levels of COL1A1 and fibronectin were measured using a QuantStudio 5 Real-Time PCR device.

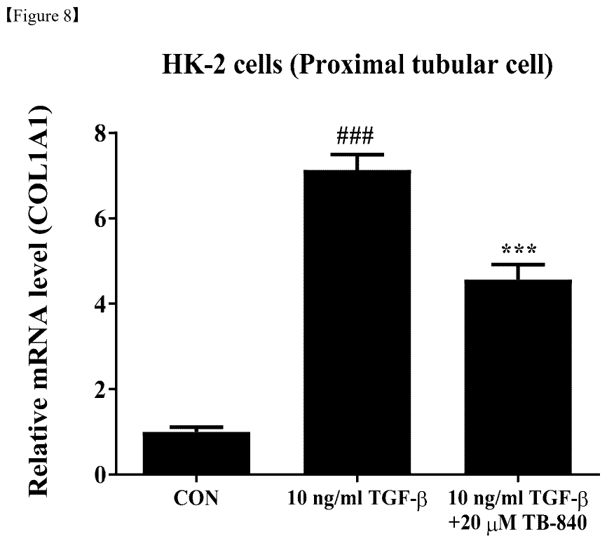

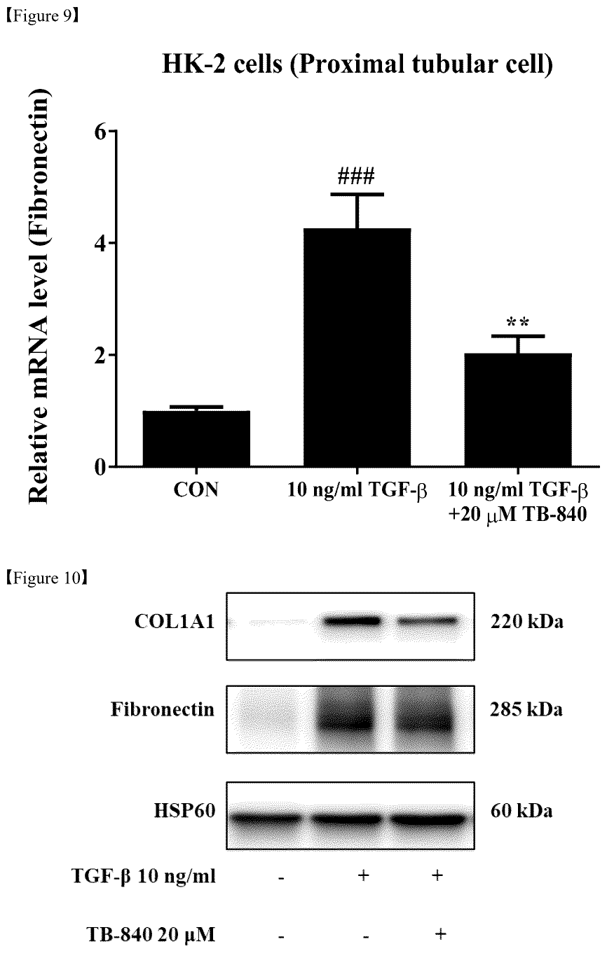

As a result, as shown in FIG. 8, the compound of the present invention was found to have reduced the gene expression of COL1A1 compared to the group treated with TGF-β at a concentration of 10 ng/ml, and as shown in FIG. 9, it was also found to have reduced the gene expression of fibronectin compared to the group treated with TGF-β at a concentration of 10 ng/ml.

Therefore, it was confirmed that the compound of the present invention inhibits the gene expression of fibrosis-related markers in kidney cells, and through this, it can be seen that it has an inhibitory effect on kidney fibrosis.

### Example 3

### Confirmation of Inhibitory Effect on Protein Expression of Fibrosis-Related Markers in Liver, Lung, and Kidney Cell Lines

In order to confirm the inhibitory effect of the compound of the present invention on the protein expression of fibrosis-related markers, the protein expression of collagen type 1 (type 1 collagen; COL1A1) and fibronectin, which are fibrosis-related markers, and HSP60 was confirmed through Western blot analysis. As a control group, TGF-β (transforming growth factor-β) was used. TGF-β is known to promote the production of extracellular matrix such as collagen and promote fibrosis of tissues.

### 3-1. Liver Cell Line (LX2)

LX2 cells were seeded at 7.6×10⁵ per dish in a 60 φ dish, cultured for 24 hours in a 37° C., 5% CO₂ incubator, and then treated with TGF-β at a concentration of 10 ng/ml and the compound of the present invention (TB-840) at a concentration of 20 μM, and cultured for 48 hours. After collecting the cultured cells, the cell pellet was spun down using a centrifuge (13,000 rpm, 5 min, 4° C.) and then lysed by adding lysis buffer with vortexing for 1 hour at 10-minute intervals, and then cell membrane components, etc. were removed using a centrifuge (13,000 rpm, 20 min, 4° C.). The sample was prepared by measuring according to the protocol using a BCA protein assay kit. Thereafter, it was denatured and separated by SDS-PAGE and transferred to a PVDF membrane. Thereafter, the primary antibody was reacted overnight, and then the secondary antibody was bound, and the protein expression of COL1A1 and fibronectin was measured using an Azure biosystems (C300) device.

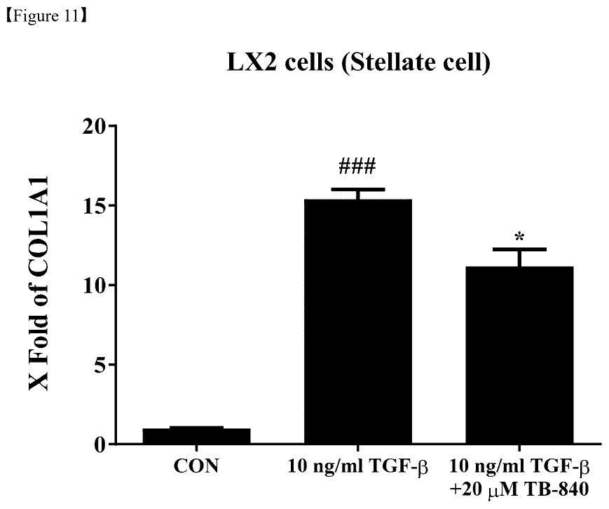

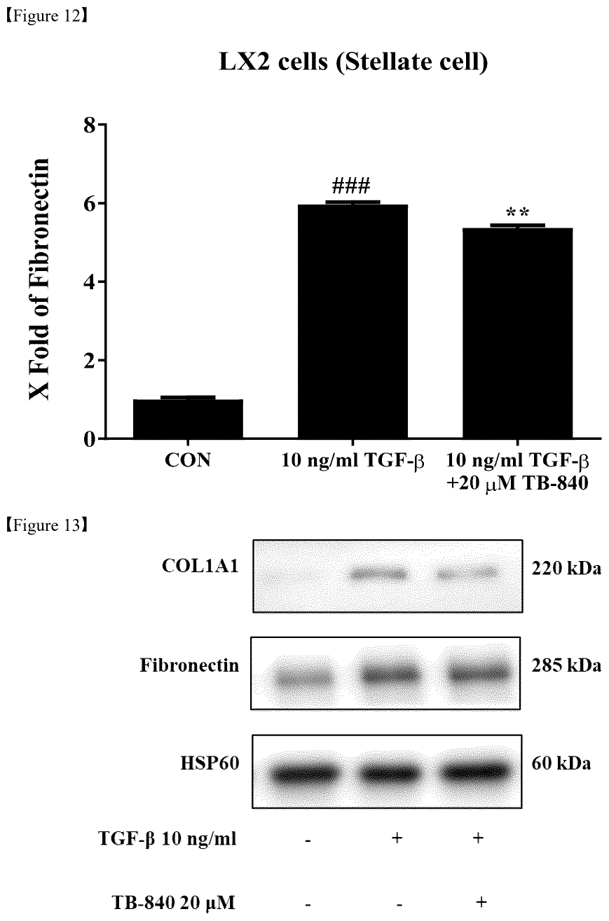

As a result, as shown in FIGS. 10 to 12, the compound of the present invention was found to have reduced the protein expression of COL1A1 and fibronectin compared to the group treated with TGF-β at a concentration of 10 ng/ml, and was found to have no change in the protein expression of HSP60.

Therefore, it was confirmed that the compound of the present invention inhibits the protein expression of fibrosis-related markers in liver cells, and through this, it can be seen that it has an inhibitory effect on liver fibrosis.

### 3-2. Lung Cell Line (A549)

A549 cells were seeded at 2.5×10⁵ per well in a 6 well plate, cultured for 24 hours in a 37° C., 5% CO₂ incubator, and then treated with TGF-β at a concentration of 10 ng/ml and the compound of the present invention (TB-840) at a concentration of 20 μM, and cultured for 24 hours. After collecting the cultured cells, the cell pellet was spun down using a centrifuge (13,000 rpm, 5 min, 4° C.) and then lysed by adding lysis buffer with vortexing for 1 hour at 10-minute intervals, and then cell membrane components, etc. were removed using a centrifuge (13,000 rpm, 20 min, 4° C.). The sample was prepared by measuring according to the protocol using a BCA protein assay kit. Thereafter, it was denatured and separated by SDS-PAGE and transferred to a PVDF membrane. Thereafter, the primary antibody was reacted overnight, and then the secondary antibody was bound, and the protein expression of COL1A1 and fibronectin was measured using an Azure biosystems (C300) device.

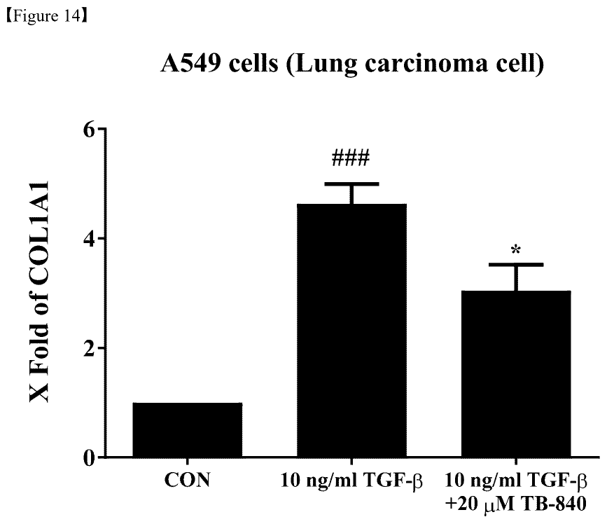

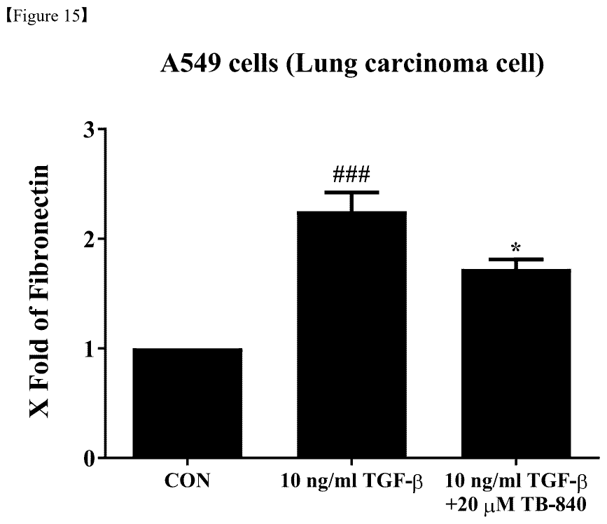

As a result, as shown in FIGS. 13 to 15, the compound of the present invention was found to have reduced the protein expression of COL1A1 and fibronectin compared to the group treated with TGF-β at a concentration of 10 ng/ml, and was found to have no change in the protein expression of HSP60.

Therefore, it was confirmed that the compound of the present invention inhibits the protein expression of fibrosis-related markers in lung cells, and through this, it can be seen that it has an inhibitory effect on pulmonary fibrosis.

### 3-3. Kidney Cell Line (HK-2)

HK-2 cells were seeded at 1×10⁶ per dish in a 60 φ dish, cultured for 24 hours in a 37° C., 5% CO₂ incubator, and then treated with TGF-β at a concentration of 10 ng/ml and the compound of the present invention (TB-840) at a concentration of 20 μM, and cultured for 48 hours. After collecting the cultured cells, the cell pellet was spun down using a centrifuge (13,000 rpm, 5 min, 4° C.) and then lysed by adding lysis buffer with vortexing for 1 hour at 10-minute intervals, and then cell membrane components, etc. were removed using a centrifuge (13,000 rpm, 20 min, 4° C.). The sample was prepared by measuring according to the protocol using a BCA protein assay kit. Thereafter, it was denatured and separated by SDS-PAGE and transferred to a PVDF membrane. Thereafter, the primary antibody was reacted overnight, and then the secondary antibody was bound, and the protein expression of COL1A1 and fibronectin was measured using an Azure biosystems (C300) device.

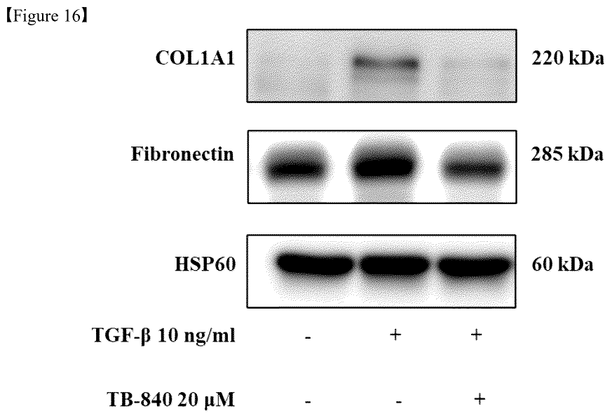

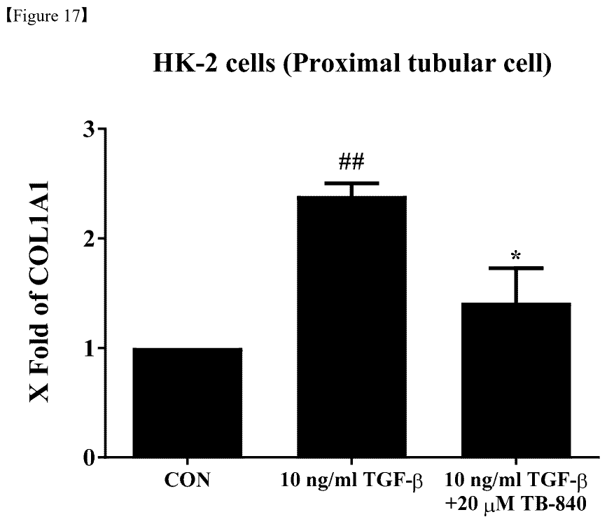

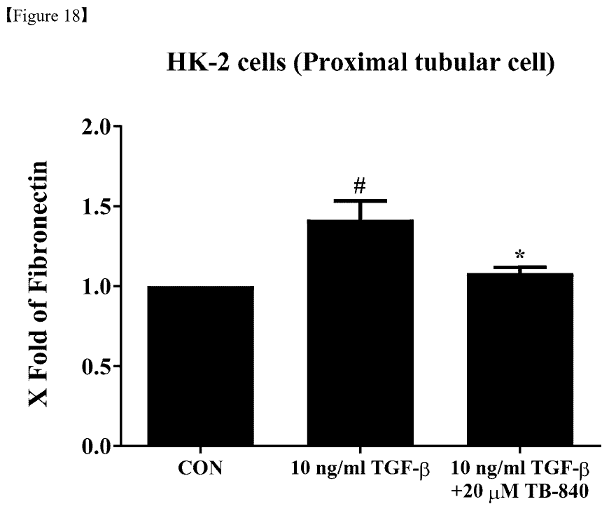

As a result, as shown in FIGS. 16 to 18, the compound of the present invention was found to have reduced the protein expression of COL1A1 and fibronectin compared to the group treated with TGF-β at a concentration of 10 ng/ml, and was found to have no change in the protein expression of HSP60.

Therefore, it was confirmed that the compound of the present invention inhibits the protein expression of fibrosis-related markers in kidney cells, and through this, it can be seen that it has an inhibitory effect on kidney fibrosis.

1. A method for preventing or treating fibrotic diseases, the method comprising administering to a subject in need thereof a compound of chemical formula 1 or a pharmaceutically acceptable salt thereof as an active ingredient, wherein the active ingredient inhibits the expression of collagen type 1 (COL1A1).

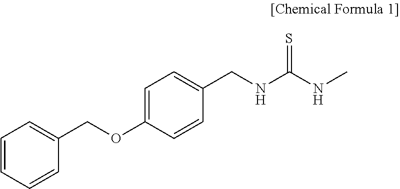 [SMILES: CNC(=S)NCc1ccc(OCc2ccccc2)cc1]

2. The method according to claim 1, wherein the fibrotic disease is selected from the group consisting of fibrosis of the lung, kidney, liver, heart, brain, blood vessels, joints, intestines, skin, soft tissue, bone marrow, penis, peritoneum, muscle, spine, testis, ovary, breast, thyroid, eardrum, pancreas, gallbladder, bladder, and prostate.

3. The method according to claim 1, wherein the fibrotic disease is liver fibrosis.

4. The method according to claim 1, wherein the fibrotic disease is pulmonary fibrosis.

5. The method according to claim 1, wherein the fibrotic disease is kidney fibrosis.

6. (canceled)

7. The method according to claim 1, wherein the active ingredient inhibits the expression of fibronectin.
